(19) United States
(12) Patent Application Publication
(10) Pub. No.: US 2024/0285868 A1
(43) Pub. Date: Aug. 29, 2024

(54) Autoinjector

(71) Applicant: Becton Dickinson France, Le Pont-de-Claix (FR)

(72) Inventors: Louis Petit, Venon (FR); Franck Carrel, Saint Jean de Vaulx (FR); Pierre Ducarouge, Grenoble (FR)

(21) Appl. No.: 18/572,923

(22) PCT Filed: Jun. 21, 2022
(86) PCT No.: PCT/EP2022/066910

§ 371 Date: Dec. 21, 2023

(30) Foreign Application Priority Data
Jun. 25, 2021 (EP) 21305882.9

(51) Int. Cl. A61M 5/315; A61M 5/20

(52) U.S. Cl. CPC A61M 5/31571; A61M 5/2033; A61M 5/31505; A61M 2005/2013

(57) ABSTRACT

This autoinjector has a proximal end and a longitudinal axis A, and a housing configured to receive a medical container having a barrel defining a reservoir for containing a medical product, said barrel having a distal end provided with a needle and an opened proximal end configured to receive a plunger rod for pushing a stopper arranged inside the barrel. A needle cover is coupled to and movable with respect to said housing between a first extended position, a retracted position, and a second extended position wherein the needle cover moves back in the distal direction to shield the needle. An injection mechanism moves the plunger rod distally inside the barrel in order to expel the medical product contained inside the barrel. A holder is movable with respect to the housing between a passive position wherein the holder does not trigger the injection mechanism and an active position wherein the holder triggers the injection mechanism. A locker is coupled to the holder, the locker being movable with regard to the housing between a locking position, wherein the locker prevents the holder from moving to the activated position, and an unlocking position, wherein the locker rotates around the longitudinal axis A, thereby allowing the holder to move to the activated position, rotation of the locker from the locking to the unlocking position being caused by the needle cover moving towards the retracted position.

### CROSS-REFERENCE TO RELATED APPLICATIONS

This application is the United States national phase of International Application No. PCT/EP2022/066910 filed Jun. 21, 2022, and claims priority to European Patent Application No. 21305882.9 filed Jun. 25, 2021, the disclosures of which are hereby incorporated by reference in their entireties.

### BACKGROUND OF THE INVENTION

### Field of the Invention

The present invention relates to an autoinjector.

In this application, the distal end of a component or of a device is to be understood as meaning the end furthest from the user's hand and the proximal end is to be understood as meaning the end closest to the user's hand. Likewise, in this application, the “distal direction” is to be understood as meaning the direction away from the user's hand, and the “proximal direction” is to be understood as meaning the direction toward the user's hand.

### Description of Related Art

Automatic injection devices are designed for automatic injection of a medical product into an injection site. Autoinjectors usually comprise a housing for receiving a medical container having a barrel defining a reservoir for containing the medical product, the barrel having a distal end provided with an injection needle and an opened proximal end receiving a plunger rod for pushing a stopper. The opened proximal end is usually provided with a flange.

Autoinjectors also include a safety shield mechanism moving from an extended to a retracted position to shield or unveil the needle and an injection mechanism for automatically injecting the medical into an injection site. The injection mechanism is usually triggered by a an activation component, also called a holder, when the needle is unveiled by the safety shield mechanism. It is important to prevent untimely triggering of the autoinjector, that is to prevent movement of the activation component towards an active position before use of the autoinjector.

To assess the robustness of the autoinjectors, autoinjectors are subjected to drop tests as required in ISO11608. These drop tests usually consist in dropping the autoinjectors at least once from a height of 1 m onto a horizontal floor. There are three drop directions, as illustrated in FIGS. 2A to 2C: a drop ‘cap upward’ (FIG. 2A), a drop ‘cap downward’ (FIG. 2B), and a drop with the autoinjector being horizontal (FIG. 2C).

During a drop ‘cap upward’, when the autoinjector strikes the ground, the accumulated kinematic energy may cause the activation component to move towards a position where it undesirably triggers the injection mechanism.

The document WO2019011688 discloses an administration assembly for a medicament delivery device.

There is therefore a need for an autoinjector that does not transition from a deactivated condition to activated condition when falling down on the floor during a drop test ‘cap upward’ or any accidental drop.

### SUMMARY OF THE INVENTION

An aspect of the invention is an autoinjector, for automatic injection of a product into an injection site, said autoinjector having a proximal end and a longitudinal axis A, the autoinjector comprising:
a housing configured to receive a medical container, said medical container having a barrel defining a reservoir for containing a medical product, and said barrel having a distal end provided with a needle and an opened proximal end configured to receive a plunger rod for pushing a stopper arranged inside the barrel,
a needle cover coupled to and movable with respect to said housing between a first extended position, wherein the needle cover at least partially shields the needle, a retracted position, wherein the needle cover moves proximally inside the housing to unshield the needle, and a second extended position wherein the needle cover moves back in the distal direction to shield the needle,
an injection mechanism configured to move the plunger rod distally inside the barrel in order to expel the medical product contained inside the barrel,
a holder movable with respect to the housing between a passive position wherein the holder does not trigger the injection mechanism and an active position wherein the holder triggers the injection mechanism,
a locker coupled to the holder, the locker being movable with regard to the housing between a locking position, wherein the locker prevents the holder from moving to the activated position, and an unlocking position, wherein the locker rotates around the longitudinal axis A, thereby allowing the holder to move to the activated position, rotation of the locker from the locking to the unlocking position being caused by the needle cover moving towards the retracted position.

Therefore, the autoinjector of the invention remains in a deactivated condition, even if the autoinjector falls down on the floor ‘proximal end first’. The autoinjector of the invention however easily permits the user who voluntarily presses the autoinjector against an injection site to move the locker towards the unlocking position and thus to trigger the injection mechanism.

The holder is axially movable along the longitudinal axis A between the passive and the active position. The proximal movement of the holder from the passive to the active position is caused by the needle cover abutting against the holder when the needle cover moves towards the retracted position. In the passive position, the holder may be axially away from a locking ring, and the locking ring is in a first position in which the locking ring prevents axial movement of the plunger rod. In the active position position, the holder abuts against the locking ring and has pushed the locking ring to a second position, in which the locking ring no longer prevents axial movement of the plunger rod.

The locker is axially movable along the longitudinal axis A between an initial position, in which the locker (and more specifically a proximal abutment surface of the locker) is axially away from the first abutment surface of the housing, and an intermediate blocking position (locking position), in which the locker (and more specifically its proximal abutment surface) abuts against the first abutment surface of the housing. The locker thus stands between the first abutment surface of the housing and the holder (more specifically a proximal shoulder of the holder). The housing blocks the locker and the locker blocks the holder in the proximal direction. Inadvertent triggering of the injection mechanism, such as during a drop “cap upward” is accordingly prevented.

The locker is further rotationally movable around the longitudinal axis A between the intermediate blocking position and a release position, in which the locker (and more specifically its proximal abutment surface) is circumferentially shifted away from the first abutment surface of the housing. After said rotation, the locker is free to move in the proximal direction and the holder is thus no longer prevented from proximally moving towards the active position.

In an embodiment, the locker has a ring portion slidingly and rotatively mounted in a groove of the holder, and a cam portion provided at a distal leg protruding from said ring portion of the locker, said cam portion being configured to abut against the needle cover when the needle cover moves towards the retracted position, thereby causing movement of the locker relative to the holder and the housing towards the unlocking position.

In an embodiment, the ring portion is in the form of a C-shaped ring portion comprising an axial slit configured to allow a snap-fit assembly of the locker onto the holder.

Preferably, the distal leg of the locker is diametrically opposite the axial slit of the ring portion of the locker.

In an embodiment, the needle cover has a cam portion configured to come in abutment against the cam portion of the locker so as to cause movement of the locker from the locking to the unlocking position.

In an embodiment, the cam portion of the locker includes a slanted wall and the cam portion of the needle cover includes an abutting edge configured to form a line contact with the locker.

In an embodiment, the abutting edge is arranged at a distal end of an axial lug, said axial lug including a lateral abutment surface configured to abut against the locker when the locker is in the unlocking position, thereby preventing the locker from moving back to the locking position.

In an embodiment, the cam portion of the needle cover includes a proximal abutment surface which is orthogonal to the longitudinal axis A so as to stop rotation of the locker and allow the needle cover to push the locker together with the holder in a proximal direction.

The proximal abutment surface may be configured such that the locker stays away from a second axial rib of the housing when the locker moves together with the holder in a proximal direction.

In an embodiment, the locker has a distal abutment surface configured to abut against a first abutment surface of the housing in the locking position, and said distal abutment surface of the locker is configured to move away from the first abutment surface of the housing when the locker is rotated towards the unlocking position.

Possibly, the first abutment surface is arranged on a first axial rib of the housing.

In an embodiment, the first abutment surface is provided at a distal end of a first axial rib of the housing.

In an embodiment, the locker has a spring mechanism for urging the locker back towards the locking position.

This permits to maintain the locker in the locking position when the autoinjector is unused.

In an embodiment, the locker is made of a single piece.

In an embodiment, the locker is made of a first material including acrylonitrile butadiene styrene (ABS) and the holder is made of a second material including polyoxymethylene (POM).

### BRIEF DESCRIPTION OF THE DRAWINGS

The terms Fig., Figs., Figure, and Figures are used interchangeably in the specification to refer to the corresponding figures in the drawings.

The invention and the advantages arising therefrom will clearly emerge from the detailed description that is given below with reference to the appended drawings as follows:

FIGS. 1A-1C are perspective views of, respectively, a first vertical drop position (cap upward), a second vertical drop position (cap downward), and a third drop position (horizontal),

FIG. 2 is a partial cross-section view of an autoinjector according to an embodiment of the invention,

FIG. 3 is a perspective view of the lower part and the upper part of an autoinjector according to an embodiment of the invention,

FIG. 4 is an exploded view of an autoinjector according to an embodiment of the invention,

FIG. 5 is a perspective view of a holder and a locker of an autoinjector according to an embodiment of the invention,

FIG. 6 is a perspective view of a locker of an autoinjector according to an embodiment of the invention,

FIG. 7 is a cross-section view in perspective of an upper housing of an autoinjector according to an embodiment of the invention,

FIG. 8A is a perspective view of a holder and a locker of an autoinjector according to an embodiment of the invention,

FIG. 8B is a cross-section view in a transversal plane of the holder and locker of FIG. 8A,

FIG. 9A is a perspective view of a holder and a locker of an autoinjector according to an embodiment of the invention,

FIG. 9B is a cross-section view in a transversal plane of the holder and locker of FIG. 9A,

FIG. 10A is a perspective view of a holder and a locker of an autoinjector according to an embodiment of the invention,

FIG. 10B is a cross-section view in a transversal plane of the holder and locker of FIG. 10A,

FIG. 10C is a cross-section view in a transversal plane of an autoinjector according to an embodiment of the invention,

FIGS. 11-23 are perspective views illustrating operating steps of an autoinjector according to an embodiment of the invention.

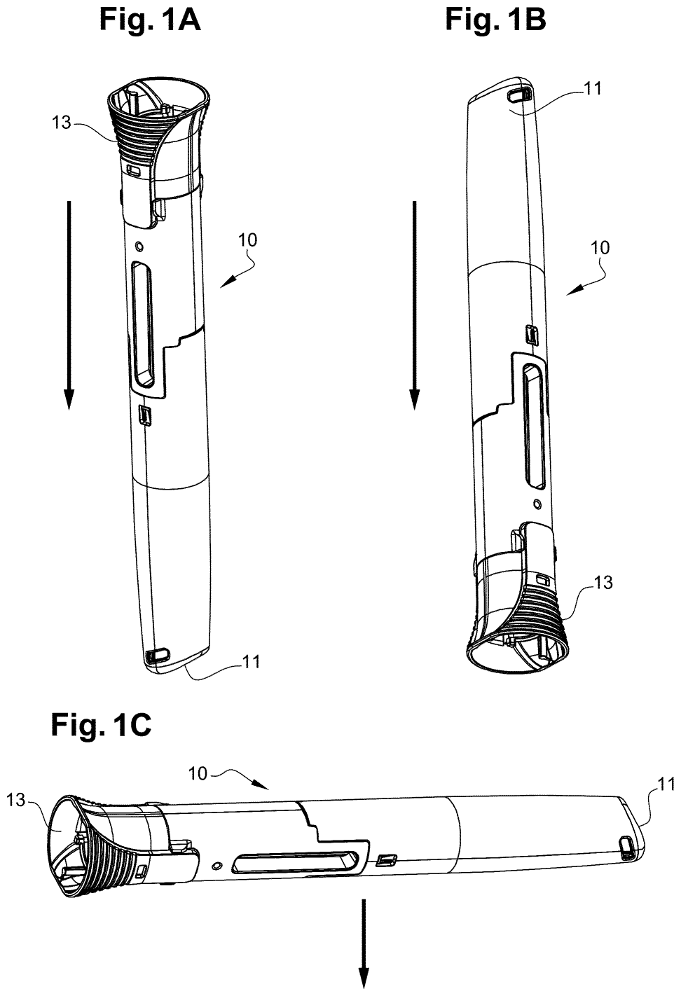

### DESCRIPTION OF THE INVENTION

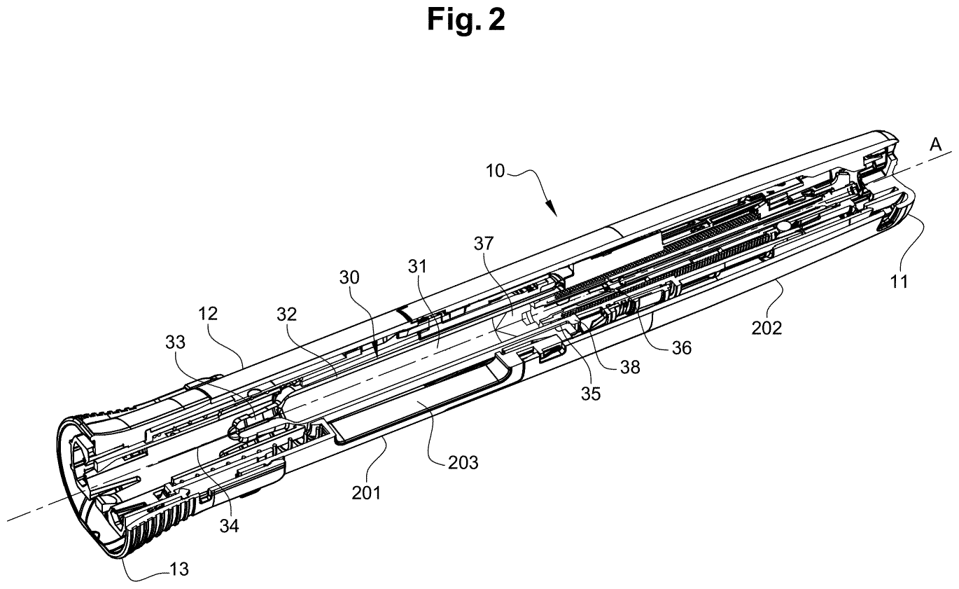

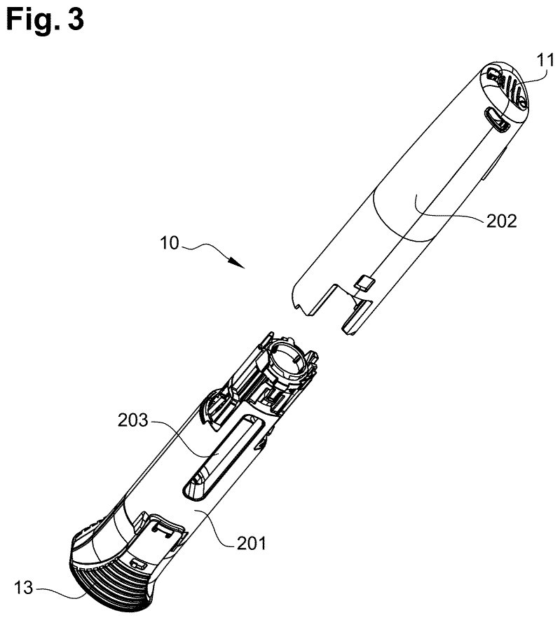

With reference to FIGS. 2 and 3 is shown an autoinjector 10 according to an embodiment of the invention. The autoinjector 10 is designed for automatic injection of a product 31 into an injection site. The autoinjector 10 comprises a housing 20 extending along a longitudinal axis A. The housing 20 is formed by the assembly of a lower housing 201 and an upper housing 202. The lower housing 201 and the upper housing 202 may be removably secured one to another by securing means such as, for instance, snap-fitting means. The autoinjector 10 has a proximal end 11 and a distal end 12. A cap 13, including a retainer 14, is removably attached to the distal end 12.

The lower housing 201 is configured to receive a medical container 30, such as a prefilled syringe. The medical container 30 has a barrel 32 defining a reservoir for containing the product 31. The lower housing 201 may define an inspection window 203 for allowing a user to visually inspect the product 31 contained within the reservoir of the medical container 30.

The barrel 32 has a distal end 33 provided with a needle 34 and an opened proximal end 35 for receiving a plunger rod 36. The plunger rod 36 may have a threaded or preferably a non-threaded distal end. The plunger rod 36 is configured to distally push a stopper 37 arranged inside the barrel 32 so as to expel the product 31 via the distal end and the needle 34. The opened proximal end 35 of the barrel 32 includes a flange 38 as illustrated in FIG. 2.

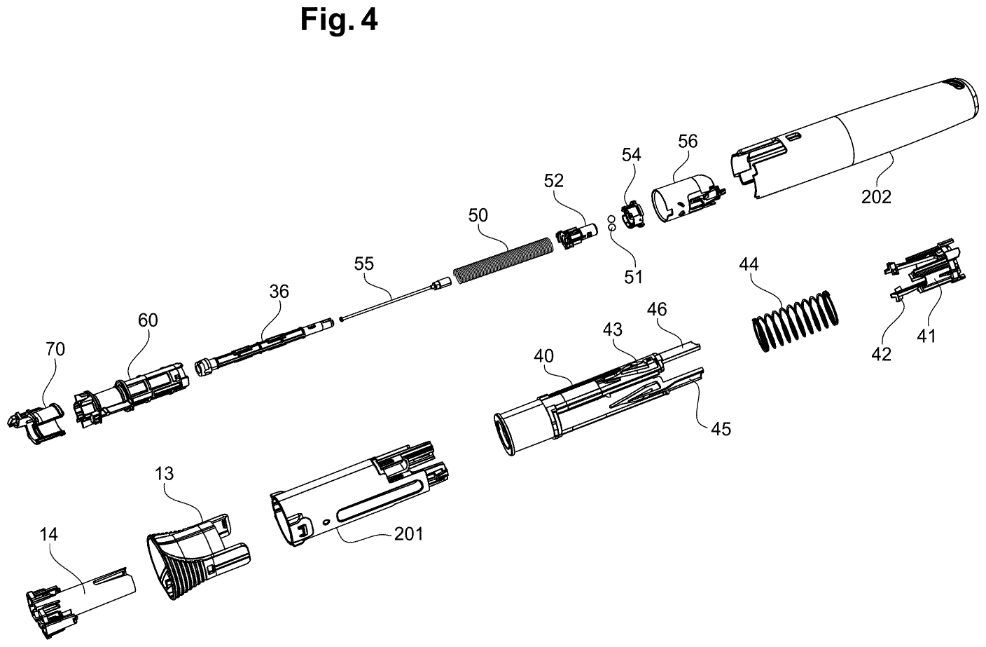

With reference to FIG. 4, the autoinjector 10 comprises a safety shield mechanism including a needle cover 40. The needle cover 40 is coupled to and movable with respect to the lower housing 201 between a first extended position (pre-use position), wherein the needle cover 40 at least partially shields the needle 34, a retracted position (injection position) wherein the needle cover 40 moves proximally inside the lower housing 201 to unshield the needle 34 (FIG. 22), thereby allowing insertion of the needle 34 into the injection site, and a second extended position (safety position), wherein the needle cover 40 moves back in the proximal direction so as to safely shield the needle 34. The safety shield mechanism includes locking means for locking the needle cover 40 in the second extended position (safety position), thereby preventing needle stick injuries. The locking means may include a locking element 41 having a proximally extending resilient leg 42 that engages a two-way slot 43 arranged through the needle cover 40. The safety shield mechanism further includes return means, such as a safety spring 44, configured to move the needle cover 40 from the retracted position to the second extended position after injection of the medical product 31 into the injection site. The needle cover 40 has a first and a second proximally extending legs 45, 46 whose function will be hereinafter described in further details.

The autoinjector 10 has an injection mechanism configured to automatically perform injection of the product 31 into the injection site. The injection mechanism includes an injection spring 50 for pushing the plunger rod 36 in the distal direction, locking balls 51 radially movable from a locking position wherein they block distal movement of the plunger rod 36 to an unlocking position wherein the locking balls 51 allow the plunger rod 36 to move in the distal direction under the action of the injection spring 50, thereby performing the injection. The injection mechanism has a washer 52 defining a radial cavity 53 for accommodating the locking balls 51, and a ring 54 coupled to and movable with respect to the washer 52 between a first position wherein the ring 54 maintains the locking balls 51 inside a recess 57 of the plunger rod 36 and a second position wherein the ring 54 allows the locking balls 51 to leave the plunger rod recess 57. The autoinjector 10 may further include a centerpiece 55 and an indicator 56 for providing the user with an audible, visual or tactile feedback that the injection is completed.

The autoinjector 10 has a tubular holder 60 configured to trigger the injection mechanism. The holder 60 is movable within the upper housing 202 from a passive to an active position. In the passive position, the holder 60 is away from the ring 54 and thus does not trigger the injection. In the activated position, the holder 60 moves the ring 54 from the first to the second position. The proximal movement of the holder 60 from the passive to the activated position is caused by the second proximal leg 46 of the needle cover 40 abutting against the holder 60 when the needle cover 40 moves from the first extended position (pre-use position) towards the retracted position (injection position).

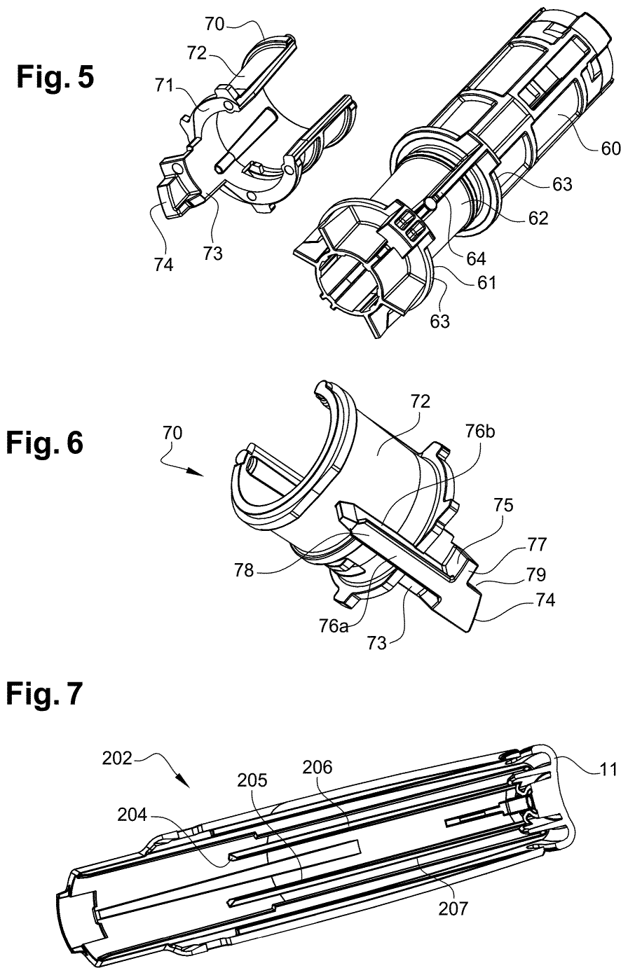

In order to prevent inadvertent movement of the holder 60 towards the active position, and thus inadvertent actuation of the injection mechanism, the autoinjector 10 includes a locker 70, preferably in the form of a C-shaped ring, as illustrated in FIGS. 5-6. To that end, the locker 70 is coupled to and movable with respect to the holder 60. The locker 70 has a distal abutment surface 71 for abutting against a proximal shoulder 61 of the holder 60. The locker 70 may slide and rotate around the longitudinal axis A with respect to the holder 60 and the upper housing 202. The holder 60 has a groove 62 for accommodating the locker 70. The groove 62 may be defined by two circumferential ribs 63 or shoulders that limit the axial movement of the locker 70 relative to the holder 60. The proximal shoulder 61 of the holder 60 may be delimited by one of these circumferential ribs 63. The groove 62 may further include an axial rib 64 that blocks the rotational movement of the locker 70 around the holder 60. In order to favor both rotational and translational movement of the locker 70 relative to the holder 60, the locker 70 may include a first material such as acrylonitrile butadiene styrene (ABS) while the holder 60 includes a second material such as polyoxymethylene (POM) that are configured to slide relative to each other with minimum friction.

More specifically, the locker 70 has a ring portion 72, that slides in the groove 62 of the holder 60, and a distal leg 73, that distally protrudes from the ring portion 72. The distal leg 73 has a cam portion 74 (at a distal end thereof), a proximal abutment surface 75, a first lateral abutment surface 76a, a second lateral abutment surface 76b, and a third lateral abutment surface 79. The cam portion 74 defines a slanted wall. The proximal abutment surface 75 may be arranged on a transversal rib 77, which may extend orthogonal to the longitudinal axis A, and which is configured to abut against a first abutment surface 204 of the upper housing 202. The first lateral abutment surface 76a, which may be formed on a side of the cam portion 74 (FIGS. 5-6) or on a side of an axial rib 78 (FIGS. 16-18), may be configured to abut against a second abutment surface 205 of the upper housing 202. The second lateral abutment surface 76b, opposite the first lateral abutment surface 76a, is configured to abut against a lateral side of a first axial rib 206 of the housing 20. The third lateral abutment surface 79, which may be formed on a side of the cam portion 74, is configured to abut against an axial lug 49 of the needle cover 40 as explained in further details below.

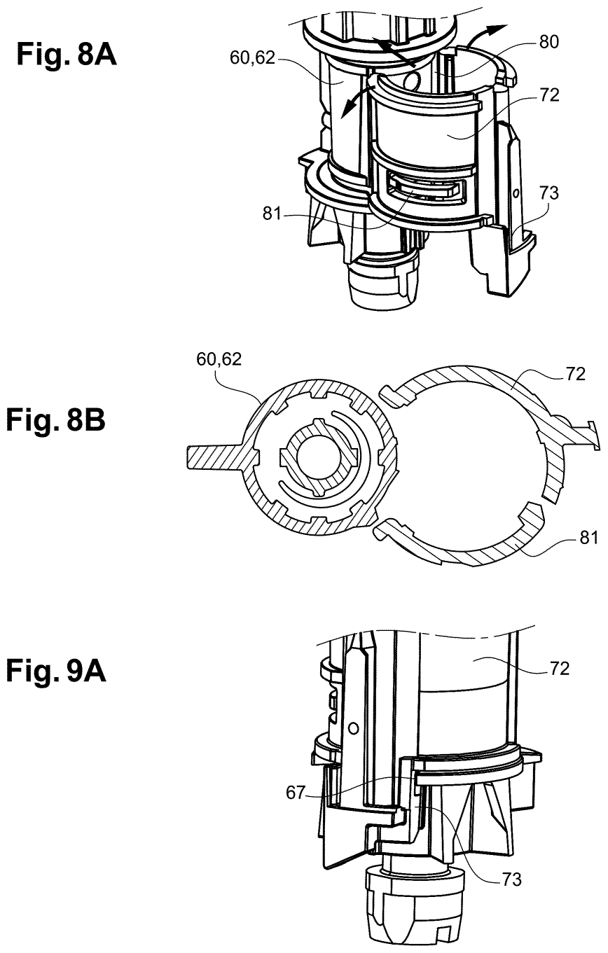

With reference to FIGS. 8A and 8B, the ring portion 72 has an axial slit 80 for allowing mounting of the locker 70 onto the holder 60. The ring portion 72 is made of a resilient material, for instance acrylonitrile butadiene styrene (ABS), so that the locker 70 may be assembled to the holder 60 by elastic deformation of the ring portion 72 as illustrated in FIG. 8A. The slit 80 of the ring portion 72 and the distal leg 73 may be diametrically opposite.

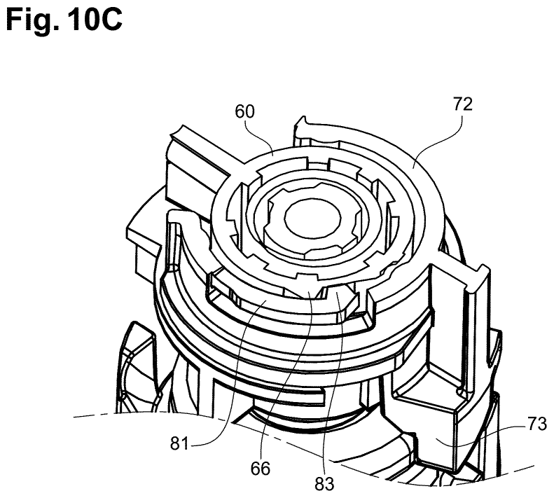

With reference to FIGS. 10A-10C, the locker 70 includes a spring mechanism, in the form of a resilient leg 81 that may have an arcuate shape. The resilient leg 81 is configured to move the locker 70 back towards the initial locking position. The resilient leg 81 extends in a circumferential slot 82 of the ring portion 72. This resilient leg 81 has a free end provided with an inward protrusion 83 configured to slide against a cam portion 66 of the holder 60 so as to recall the locker 70 back towards the initial position.

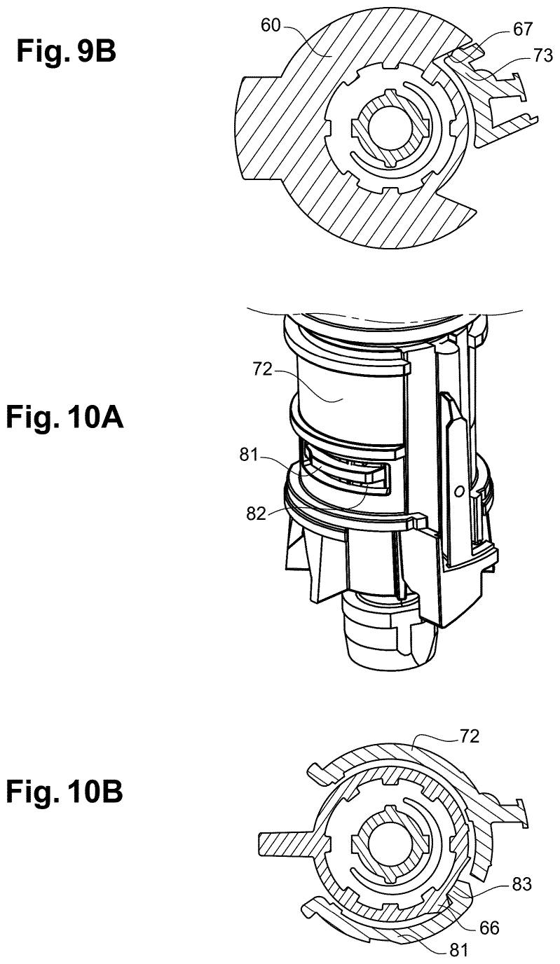

With reference to FIGS. 9A-9B, the holder 60 has a lateral abutment surface 67 configured to abut against the distal leg 73 for maintaining the locker 70 in an initial position relative to the holder 60 before assembly of the autorinjector 10. However, when the holder 60 and the locker 70 are mounted in the upper housing 202, the resilient leg 81 causes the locker 70 (more specifically its second lateral abutment surface 76b) to abut against the upper housing 202 (more specifically against a lateral surface of an axial rib 206).

It is noted that the locker 70 is preferably made of a single piece.

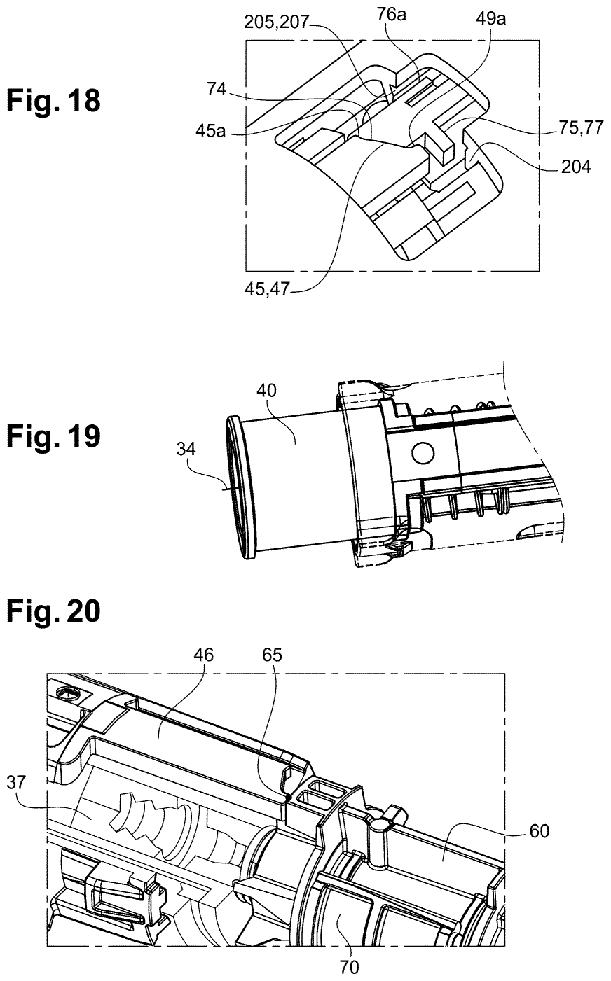

Both translation and rotation of the locker 70 are caused by the first proximal leg 45 of the needle cover 40 abutting against the cam portion 74 of the locker 70. As illustrated in FIGS. 14-18, the first proximal leg 45 of the needle cover 40 may have a cam portion 47 at its proximal end. This proximal end of the needle cover 40 and the distal end of the locker distal leg 73 may be complementarily shaped. With reference to FIGS. 14-18, the cam portion 47 of the first proximal leg 45 of the needle cover 40 may have an abutting edge 48 configured to come in abutment against the slanted wall of the locker cam portion 74. The abutting edge 48 extends in a radial direction and permits to establish a line contact between the needle cover 40 and the locker 70. This line contact causes rotation of the locker 70 due to the proximal movement of the needle cover 40 with minimum friction interence between the needle cover 40 and the locker 70. Thus, activation of the autoinjector 10 does not require the user to exert a high pressure force against the injection site. The abutting edge 48 may be located on an axial lug 49 that extends in the proximal direction from the proximal-most part of the needle cover cam portion 47. The axial lug 49 advantageously defines a lateral abutment surface 49a (FIG. 18) configured to abut against the distal leg 73 of the locker 70. As a result, the locker 70 is prevented to rotate back to its initial locking position when the blocking surface abuts against the distal leg 73, as illustrated in FIG. 18.

Still with reference to FIG. 18, the first proximal leg 45 of the needle cover 40 may have a proximal abutment surface 45a configured to abut against the distal-most part of the locker cam portion 74. This proximal abutment surface 45a is preferably orthogonal to the longitudinal axis A and permits to stop rotation of the locker 70 caused by the needle cover 40.

Between the axial lug 49 and the proximal abutment surface 45a, the cam portion 47 of the needle cover 40 may have a slanted wall having the same shape as the slanted wall of the locker cam portion 74.

With reference to FIG. 7, the upper housing 202 may have one or several first axial ribs 206 and one or several second axial ribs 207, extending along the longitudinal axis A and protruding from an inner lateral wall of the upper housing 202. The first axial rib 206 defines a first abutment surface 204, which may be arranged at a distal end of this first axial rib 206. The first abutment surface 204 is configured to stop a proximal movement of the locker 70, by abutting against the proximal abutment surface 75 of the locker 70, and thus temporarily block the locker 70 in an intermediate blocking position wherein the locker 70 prevents the holder 60 from moving towards the active position. The second axial rib 207 has a second abutment surface 205, which may be arranged at one side of the second axial rib 207, which may serve to stop rotation of the locker 70 by abutting against the first lateral abutment surface 76a of the locker 70, if the needle cover 40 is devoid of the proximal abutment surface 45a or the locker 70 is devoid of resilient leg 81.

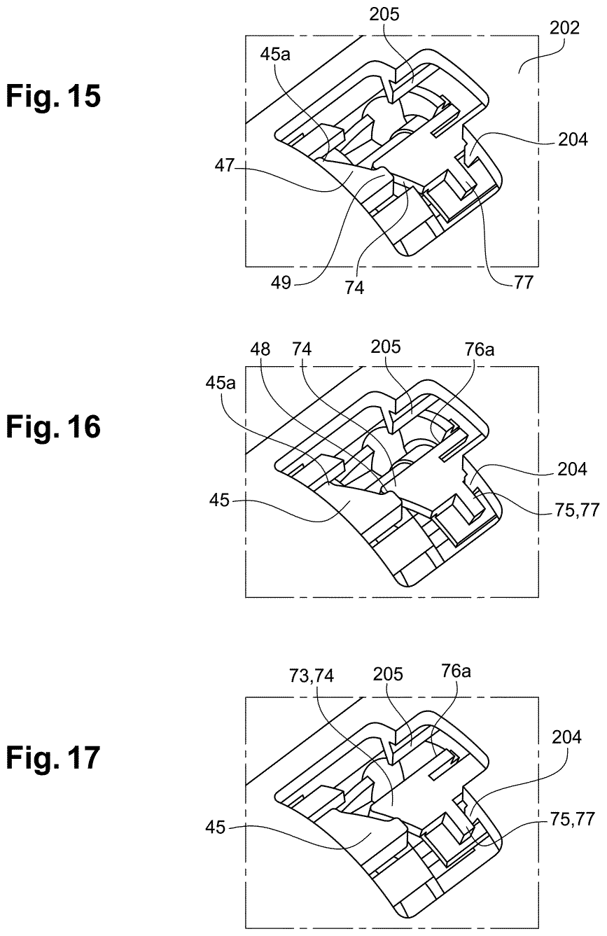

The locker 70 is movable between an initial position (FIG. 15) wherein the locker 70 is away from the first abutment surface 204 of the upper housing 202, an intermediate blocking position (FIG. 16) wherein the locker 70 moves proximally and abuts against the first abutment surface 204 of the upper housing 202, and a release position (FIG. 18) wherein the locker 70 rotates away from the first axial rib 206. In the intermediate blocking position, the locker 70 prevents the holder 60 from being moved towards the activated position, because the proximal shoulder 61 of the holder 60 would abut against the distal abutment surface 71 of the locker 70 and the locker 70 abuts against the first abutment surface 204 of the upper housing 202. Thus, the autoinjector 10 remains in a deactivated condition. Rotation of the locker 70 from the temporary blocking position to the release position is preferably stopped by the proximal abutment surface 45a of the needle cover 40 abutting against the cam portion 74 of the locker 70, instead of being stopped by the second axial rib 207 of the upper housing 202. Accordingly, in the release position, the locker 70 is preferably maintained by the resilient leg 81 against the lateral abutment surface 49a of the needle cover 40 instead of abutting against the second axial rib 207. This avoids friction forces between said second axial rib 207 and the locker 70. In the release position (i.e. after rotation), the locker 70 is free to move proximally and accordingly allows the holder 60 to move proximally towards the activated position. In the release position of the locker 70, the autoinjector 10 is ready for activation. The autoinjector 10 is activated (the injection mechanism is triggered) when the holder 60 reaches the activated position.

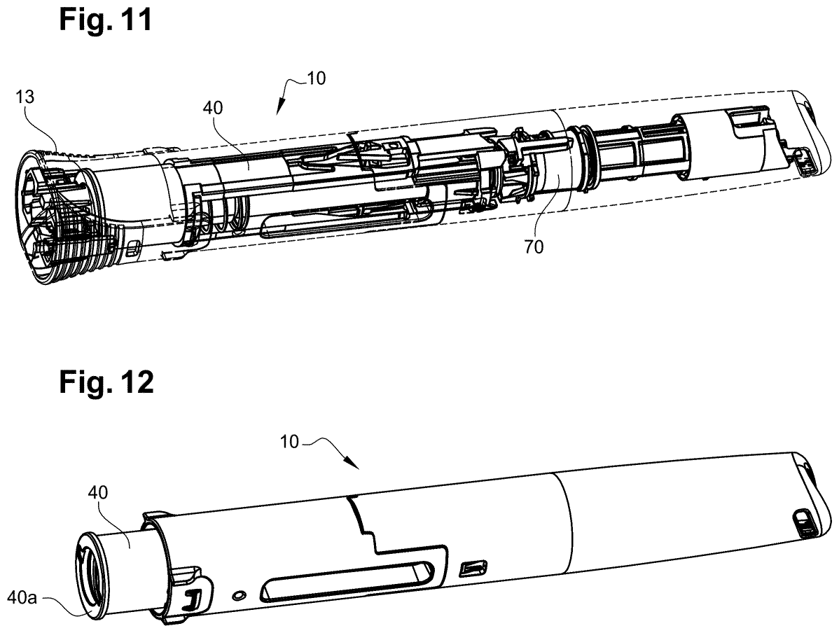

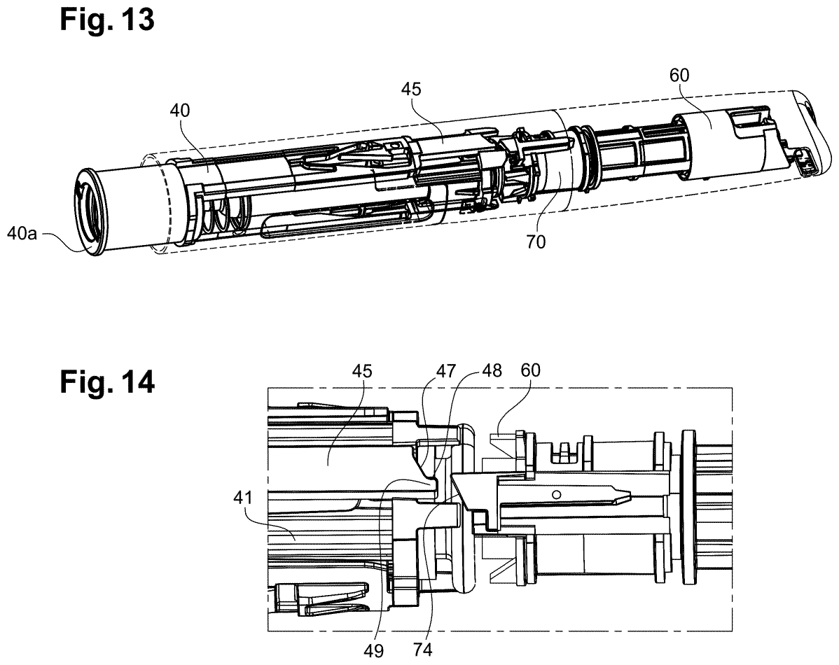

The operation of the autoinjector 10 according to the invention is described below in connection with FIGS. 11-23.

With reference to FIGS. 11-14, the user firstly removes the cap 13 and applies a distal end 40a of the needle cover 40 against an injection site. At this stage, the first proximal leg 45 of the needle cover 40 is distally away from the cam portion 74 of the locker 70, and the locker 70 is maintained by the resilient leg 81 in an initial position wherein its proximal abutment surface 75 faces the first abutment surface 204 of the first axial rib 206.

The user presses the autoinjector 10 against the injection site. This causes the needle cover 40 to move in the proximal direction towards the retracted position and thus come in abutment against the cam portion 74 of the locker 70 (FIG. 15). Further movement of the needle cover 40 causes the locker 70 to move proximally along the longitudinal axis A and results in the locker 70 abutting against the first abutment surface 204 (FIG. 16).

Here, the locker 70 is in the intermediate blocking position and accordingly prevents the holder 60 from inadvertently moving to the active position. If movement of the needle cover 40 was caused by a drop ‘cap upward’ of the autoinjector 10, then nothing happens, the autoinjector 10 safely remains in the deactivated condition by means of the locker 70 abutting against the housing 20. If, however, movement of the needle cover 40 is caused by the user pressing the autoinjector 10 against an injection site, then the locker 70 begins to rotate as the user goes on pushing the autoinjector 10.

Indeed, due to the shape of the locker cam portion 74 and the needle cover cam portion 47, and due to the abutment between the locker 70 and the upper housing 202, further movement of the needle cover 40 in the proximal direction causes the locker 70 to rotate around the holder 60 (FIG. 16).

The locker 70 rotates until the proximal abutment surface 45a of the needle cover 40 abuts against the cam portion 74 of the locker 70. The locker 70 is maintained by the resilient leg 81 against the lateral abutment surface 49a of the axial lug 49 which abuts against a side of the distal leg 73 of the locker 70 (FIG. 18). Preferably, the locker 70 stays away from the second axial rib 207 so as to ease the subsequent proximal movement of the holder 60 towards the active position. The locker 70 is in the release position: the locker 70 no more prevents the holder 60 from moving in the proximal direction. The autoinjector 10 is ready for being activated.

At that time, the needle cover 40 may unveil the needle 34 and the needle 34 may begin insertion into the injection site (FIG. 19).

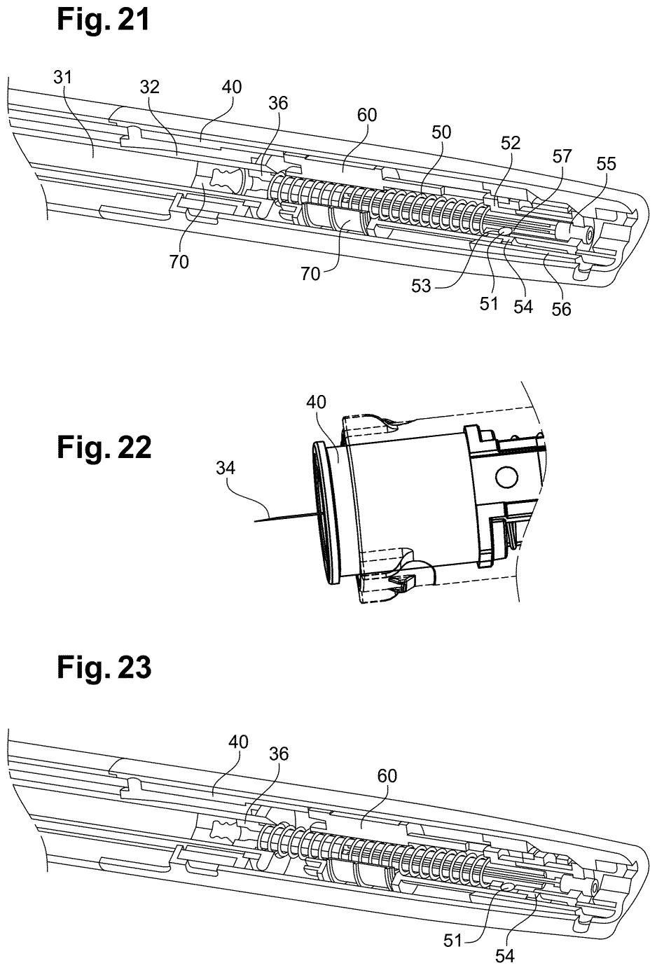

The second proximal leg 46 of the needle cover 40 comes against a distal end 65 of the holder 60 (FIG. 20) and pushes the holder 60 in the proximal direction (FIG. 21). The holder 60 leaves the passive position and progresses towards the activated position.

At this stage, the locking balls 51 extend in the locking recess of the plunger rod 36 and thus prevent the injection spring 50 from pushing the plunger rod 36 in the distal direction. The locking ring is in the first position such that the locking ring blocks the locking balls 51 inside the locking recess of the plunger rod 36.

However, when the needle cover 40 reaches the retracted position (injection position), as illustrated in FIGS. 22 and 23, the holder 60 reaches the activated position. The holder 60 comes in abutment against the locking ring and moves the locking ring in the proximal direction to the second position. The locking balls 51 are hence free to move radially outwardly, outside the locking recess of the plunger rod 36. As a result, the plunger rod 36 is no more maintained by the locking balls 51 and the injection spring 50 extends, moving the plunger rod 36 in the distal direction. The plunger rod 36 then pushes the stopper 37 inside the barrel 32, thereby expelling the product 31 through the needle 34 into the injection site. Injection is triggered, and the autoinjector 10 is activated.

At the end of the injection, the centerpiece 55 cooperates with the plunger rod 36 and the indicator to provide the user with a feedback indicating that the injection is completed.

The user may take the autoinjector 10 away from the injection site. The safety spring 44 accordingly moves the needle cover 40 back in the distal direction, until the needle cover 40 reaches the second extended position (safety position) wherein the needle cover 40 shields the needle 34 and is prevented from moving back in the retracted position by the locking element 41.

Due to the locker 70, the invention permits to prevent inadvertent activation of the autoinjector 10, for example if the autoinjector 10 falls down on the floor, while still permitting easy activation of the autoinjector 10 when this activation results from the user intentional action.

1. An autoinjector, for automatic injection of a product into an injection site, said autoinjector having a proximal end and a longitudinal axis A, the autoinjector comprising:
a housing configured to receive a medical container, said medical container having a barrel defining a reservoir for containing a medical product, and said barrel having a distal end provided with a needle and an opened proximal end configured to receive a plunger rod for pushing a stopper arranged inside the barrel,
a needle cover coupled to and movable with respect to said housing between a first extended position, wherein the needle cover at least partially shields the needle, a retracted position, wherein the needle cover moves proximally inside the housing to unshield the needle, and a second extended position wherein the needle cover moves back in the distal direction to shield the needle,
an injection mechanism configured to move the plunger rod distally inside the barrel in order to expel the medical product contained inside the barrel,
a holder movable with respect to the housing between a passive position wherein the holder does not trigger the injection mechanism and an active position wherein the holder triggers the injection mechanism,
a locker coupled to the holder, the locker being rotationally movable with regard to the housing, between a locking position, in which a proximal abutment surface of the locker abuts against a first abutment surface of the housing so that the locker prevents the holder from moving to the activated position, and an unlocking position, in which the proximal abutment surface of the locker is moved away from the first abutment surface of the housing, thereby allowing the holder to move to the activated position, rotation of the locker from the locking to the unlocking position being caused by the needle cover moving towards the retracted position.

2. The autoinjector according to claim 1, wherein the locker has a ring portion slidingly and rotatively mounted in a groove of the holder, and a cam portion provided at a distal leg protruding from said ring portion of the locker, said cam portion being configured to abut against the needle cover when the needle cover moves towards the retracted position, thereby causing movement of the locker relative to the holder and the housing towards the unlocking position.

3. The autoinjector according to claim 1, wherein the ring portion is in the form of a C-shaped ring portion comprising an axial slit configured to allow a snap-fit assembly of the locker onto the holder.

4. The autoinjector according to claim 2, wherein the needle cover has a cam portion configured to come in abutment against the cam portion of the locker so as to cause movement of the locker from the locking to the unlocking position.

5. The autoinjector according to claim 4, wherein the cam portion of the locker includes a slanted wall and the cam portion of the needle cover includes an abutting edge configured to form a line contact with the locker.

6. The autoinjector according to claim 5, wherein the abutting edge is arranged at a distal end of an axial lug, said axial lug including a lateral abutment surface configured to abut against the locker when the locker is in the unlocking position, thereby preventing the locker from moving back to the locking position.

7. The autoinjector according to claim 4, wherein the cam portion of the needle cover includes a proximal abutment surface which is orthogonal to the longitudinal axis A so as to stop rotation of the locker and allow the needle cover to push the locker together with the holder in a proximal direction.

8. The autoinjector according to claim 1, wherein the first abutment surface is provided at a distal end of a first axial rib of the housing.

9. The autoinjector according to claim 1, wherein the locker has a spring mechanism for urging the locker back towards the locking position.

10. The autoinjector according to claim 1, wherein the locker is made of a single piece.

11. The autoinjector according to claim 1, wherein the locker is made of a first material including acrylonitrile butadiene styrene (ABS) and the holder is made of a second material including polyoxymethylene (POM).
